(19) United States
(12) Patent Application Publication
(10) Pub. No.: US 2007/0004982 A1
(43) Pub. Date: Jan. 4, 2007

(54) Apparatus and method for early detection of cardiovascular disease using vascular imaging

(75) Inventors: Thomas Hugh Marwick, Wooloongabba (AU); Brian Haluska, Wooloongabba (AU)

Correspondence Address: HARNESS, DICKEY & PIERCE, P.L.C., P.O. BOX 828, BLOOMFIELD HILLS, MI, 48303, US

(73) Assignee: The University of Queensland, ST. Lucia, Queensland (AU)

(21) Appl. No.: 10/567,137

(22) PCT Filed: Aug. 5, 2004
(86) PCT No.: PCT/AU04/01041

§ 371 Date: May 23, 2006

(30) Foreign Application Priority Data
Aug. 5, 2003 (AU) 2003904100

(51) Int. Cl. A61B 8/00

(52) U.S. Cl. 600/437

(57) ABSTRACT

The invention provides an apparatus (10) and method (24) for determining vascular characteristics for early detection of cardiovascular disease comprising: an ultrasonic signal source (32) directing ultrasound signals (30) at an artery (27); an ultrasonic signal receiver (34) receiving ultrasound signals (36) reflected from or transmitted through the artery (27); means for analysing signals (40) received by ultrasonic signal receiver (34) to extract arterial displacement data (42); means for acquiring blood pressure data (48); signal processing means (44) for adjusting said arterial displacement data (46) using the blood pressure data (48); and means for analysing the adjusted arterial displacement data (51) to characterise vascular function (28).

### FIELD OF THE INVENTION

The present invention broadly relates to a method and apparatus for detecting early cardiovascular disease. In particular, this invention relates to an apparatus and method utilising vascular imaging techniques.

### BACKGROUND OF THE INVENTION

Cardiovascular disease (CVD) is the leading cause of disability and death in the western world, resulting in more premature deaths than any other illness. Unsurprisingly, treatment of CVD represents the highest cost burden to any healthcare system. Accordingly, there is tremendous social and political pressure to develop earlier and more reliable diagnostic tests to assist in the detection, treatment and prevention of CVD.

Changes in the structure and function of blood vessels are known to be an early stage indicator in the development of CVD. This suggests that tests of vascular function may be used to diagnose early disease and track the response to various treatments that cause disease regression.

Coronary angiography and stress testing, which have been the cornerstone of the diagnosis and management of coronary artery disease, are ineffective in diagnosing early sub-clinical disease because they depend on the detection of luminal narrowing, while early disease causes vessel expansion. Although angiography may be used to identify earlier lesions, it does not directly assess the vessel wall unless intravascular ultrasound is performed. This is invasive and expensive. Various invasive techniques have been used to examine endothelial function in patients with coronary artery disease. However, these are poorly suited to sequential follow-up, and being invasive, carry the potential of significant adverse effects.

The most widely used non-invasive testing is brachial artery reactivity (Celermajer D S et al., Lancet 1992; 340:1111-5). However, in using this method, the measurement of flow-mediated vasodilation is technically challenging. Normal ranges show large standard deviations, in part because the results are influenced by a number of acute stimuli, including the fasting state, tobacco, caffeine and vasoactive drug loads. Unfortunately, the presence of both vascular disease and risk factors influence the result.

Another technique which has been developed is applanation tonometry (Hayward C S et al. Hypertension, 2002; 40:e8-e9). This non-invasive clinical tool measures the elastic properties of the entire arterial tree, reflecting systemic vascular changes. Applanation tonometry uses a transcutaneously-applied micromanometer tipped probe which is placed against an arterial wall. When there is sufficient pressure to distort, or applanate the artery, it creates a signal that approximates instantaneous arterial pressure. The signal is then digitised and reconstructed on a PC. This application is most feasible over distal vessels, such as the radial artery with minimal soft tissue cover and an underlying bony surface to support it, rather than over the proximal vessels, eg. the carotid arteries, which are embedded in adipose tissue and muscle and do not have the same support structure and therefore are subject to movement and subtle pressure changes. While central aortic pressure is assumed to be equal to carotid pressure due to the proximity of the vessels, carotid tonometry is technically challenging and suffers from test-retest variability. Although the radial technique is less limited by these problems, the use of a transfer function to reconstruct a central waveform may be particularly problematic in the elderly or women. A further limitation is that medical specialists who are most likely to use the data are unfamiliar with the technology. Applanation tonometry requires specialist equipment and training, which have both compromised the uptake of the technique.

Another non-invasive method is total arterial compliance (TAC) (eg. Segers et al. Ann Biomed Eng 1999; 27:480-5). TAC measures systemic distensibility based on the pulse-pressure method derived from the two-element Windkessel model, i.e. the increment of volume of the systemic arterial bed for an increment in distending pressure of the entire systemic arterial tree. Compliance falls with the loss of elastic function in the great vessels, as occurs in conditions such as hypertension and atherosclerotic vascular disease. Several approaches have been used to measure TAC. One such technique requires simultaneous measurement of stroke volume and arterial pressure, with the TAC value (mls/mmHg) being derived mathematically from three separate measurements: tonometry for pressure, 2D echo for orifice area and Doppler for flow.

In recognising the need for non-systemic direct measurement of vessel wall displacement, techniques using M-mode (Gamble et al. Stroke 1994; 25(1): 11-16) and radiofrequency signals (Hoeks et al, Ultrasound Med Biol 1990; 16(2): 121-8) have been explored. However both techniques have shown to be highly complex and highly dependent on two-dimensional image quality when used clinically.

Another method, Doppler echocardiography is used traditionally to evaluate the velocity and direction of blood flow in the heart and vessels. Recent technical developments have allowed reduction of the wall filters and scale, thus permitting the evaluation of low velocity, high amplitude signals which come from tissue. Colour tissue Doppler imaging (TDI) is a technique in which the velocity of myocardial movement toward the transducer is displayed in colour-coded form on myocardial images. Advantageously, this technique permits rapid, simultaneous visualisation of several walls, either myocardial or vascular, in a single view. However, this method does not (i) provide for means of assessing local vascular behaviour, but rather systemic measurements or (ii) consider the influencing factors of distensibility or blood pressure.

There exists a need for the development of a simple, accurate means of assessing direct or local vascular elasticity that will allow for early detection of arterial disease and will provide a tool for monitoring outcomes of treatment and preventive medicine.

### OBJECT OF THE INVENTION

Accordingly, it is an object of the invention to provide an apparatus and method using Doppler imaging to overcome one or more of the problems of the prior art or provide a useful commercial alternative.

### SUMMARY OF THE INVENTION

According to the present invention there is provided a method for determining vascular characteristics for early detection of cardiovascular disease including the steps of:

(i) acquiring velocity displacement data from arterial colour tissue Doppler imaging;

(ii) processing the velocity displacement data to generate arterial displacement data;

(iii) adjusting the arterial displacement data using blood pressure data; and

(iv) analysing the adjusted arterial displacement data to characterise vascular function.

Preferably, the step of processing the velocity displacement data includes integrating velocity displacement data with respect to time.

More preferably, the step of processing the velocity displacement data includes using a readable spreadsheet for integrating velocity displacement data with respect to time.

Suitably, the step of adjusting the arterial displacement data includes using mean and diastolic brachial cuff blood pressure data.

More suitably, the step of adjusting the arterial displacement data includes using mean and diastolic brachial cuff blood pressure data when acquired by a manometer.

In one embodiment, the step of analysing the adjusted arterial displacement data includes generating local elasticity data.

Preferably, the step of generating local elasticity data includes correcting the observed arterial displacement data for pressure by dividing the observed displacement data by the log of the pulse pressure obtained from cuff blood pressure.

In an alternative embodiment, the step of analysing the adjusted arterial displacement data includes generating central blood pressure data.

Preferably, the step of generating central blood pressure data includes calibrating the adjusted arterial displacement data from the mean and diastolic blood pressure obtained from cuff blood pressure to reflect pressure over time.

According to a second aspect of the present invention there is provided an apparatus for determining vascular characteristics for early detection of cardiovascular disease comprising:

an ultrasonic signal source directing ultrasound signals at an artery;

an ultrasonic signal receiver receiving ultrasound signals reflected from or transmitted through the artery;

means for analysing signals received by ultrasonic signal receiver to extract arterial displacement data;

means for acquiring blood pressure data;

signal processing means for adjusting said arterial displacement data using the blood pressure data; and

means for analysing the adjusted arterial displacement data to characterise vascular function.

Preferably, the means for analysing signals received by ultrasonic signal receiver includes means for integrating velocity displacement data with respect to time.

Suitably, the means for acquiring blood pressure data includes a means for measuring diastolic and mean brachial cuff blood pressure data.

More suitably, the means for acquiring blood pressure data includes a manometer for measuring diastolic and mean brachial cuff blood pressure data.

Preferably, the signal processing means includes means for adjusting arterial displacement data with respect to blood pressure data.

In one embodiment, the means for analysing the adjusted arterial displacement data includes a means of generating vascular function data in the form of local elasticity data.

Preferably, the means of generating local elasticity data includes a means for correcting pressure-adjusted displacement data by dividing the arterial displacement data by the log of the cuff blood pressure.

In another embodiment, the means for analysing the adjusted arterial displacement data includes a means of generating vascular function data in the form of central blood pressure data.

Preferably, the means of generating central blood pressure data includes a means for generating a calibrated curve that reflects pressure over time.

### BRIEF DESCRIPTION OF THE DRAWINGS

In order that the present invention may be more readily understood and placed into practical effect, preferred embodiments of the invention will be described, by way of example only, with reference to the accompanying drawings, in which:

FIG. 1 is a diagram of the method of the invention showing the steps for the generation of vascular function data.

FIG. 2 is a schematic diagram of an apparatus for early detection of cardiovascular disease using the arterial imaging method of FIG. 1.

FIG. 3 shows the output from the analysed arterial colour tissue Doppler with raw tissue Doppler (lower left), individual displacement curves for each cardiac cycle (top), and mean displacement curve (bottom right).

FIG. 4 shows the output from Samtdi analysis program with raw displacement curves (upper left), raw carotid tonometry (lower left), and a comparison of calibrated displacement curves and tonometry (right).

FIG. 5 shows that arterial displacement corrected for pressure reduces as the degree of arterial disease increases in a patient study.

FIG. 6 is a comparison of displacement between two patients in a study; one having high arterial displacement at a low blood pressure (left); and one having lower displacement at a much higher pressure (right).

FIG. 7 shows the same inverse relationship between total arterial compliance and displacement as arterial disease progresses.

FIG. 8 shows the inverse relationship between carotid intima-media thickness and displacement in the study of FIG. 7; as IMT increases with arterial disease, displacement decreases.

FIG. 9 shows the relationship with brachial artery reactivity, or the ability of the artery to dilate in response to hyperaemia, to progressing arterial disease.

FIG. 10 is a Bland-Altman plot showing strong correlation and differences between pressures obtained from carotid tonometry and calibrated TDI for systolic blood pressure.

### DETAILED DESCRIPTION OF THE PREFERRED EMBODIMENTS

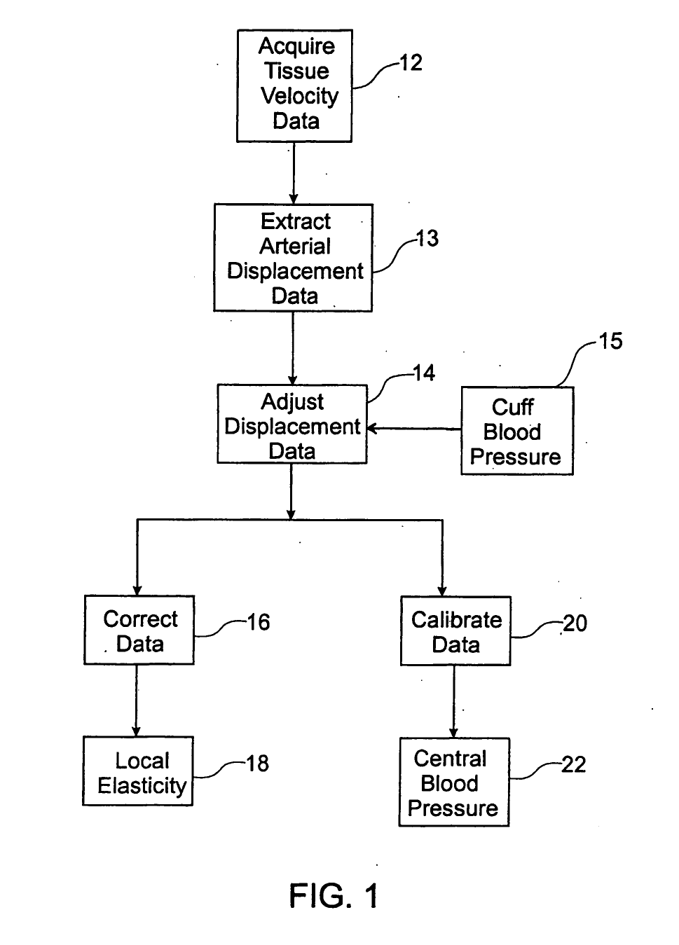

Referring to FIG. 1, the method 10 of generating characteristic vascular function is broadly described. The initial step of acquiring tissue velocity data 12 from arterial colour tissue Doppler imaging is followed by the subsequent extraction of “observed” arterial displacement data 13 from the velocity data 12. Cuff blood pressure (BP) data 15 is acquired and used in the adjustment 14 of the arterial displacement data 13. Preferably, the blood pressure data 15 used is diastolic and mean brachial cuff blood pressure.

The method 10 of the present invention provides a means to measure the vascular function characteristics of both local arterial elasticity and central blood pressure.

The adjusted displacement data 14 is analysed to generate corrected displacement data 16, which in turn generates local elasticity data 18. By ‘corrected displacement’ is meant a sound approximation of local elasticity. The corrected displacement data 16 are generated by dividing the observed displacement data by the log of the pulse pressure obtained from cuff sphygmomanometry, or cuff BP 15 to give a pressure-adjusted displacement value. The log of the pulse pressure is used to adjust for the non-linear nature of the pulse pressure. This may be carried out conveniently using a software-based readable spreadsheet.

In an alternative embodiment, the adjusted displacement data 14 may be calibrated 20 to generate central blood pressure data 22. In use, calibrating the adjusted arterial displacement data 14 from the mean and diastolic blood pressure is obtained from cuff sphygmomanometry or cuff BP 15, the calibrated curve reflecting pressure over time. As for above, this may be carried out conveniently using a software-based readable spreadsheet.

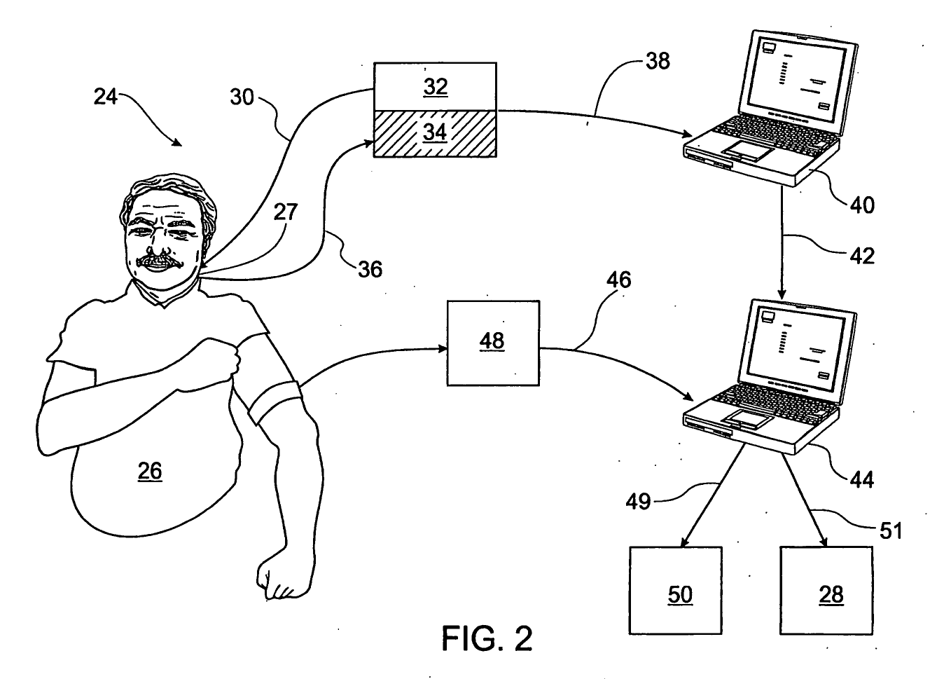

FIG. 2 shows the early detecting CVD apparatus 24. In use, the apparatus 24 is connected to a patient 26 to measure waveform velocity data 28 as a measure of the local arterial elasticity. Specifically, the velocities derived from the smooth muscle layer as the artery expands in systole and contracts in diastole are used to calculate arterial displacement, which is a measure of arterial elasticity 28. Tissue Doppler imaging data or arterial velocity displacement data 38 are acquired by directing ultrasound signals 30 at an artery of a patient 27 using an ultrasonic signal source 32. An ultrasonic signal receiver 34 receives ultrasound signals 36 that are reflected from or transmitted through the carotid artery of the patient 27.

The signals 36 received by the ultrasonic signal receiver 34 are analysed to extract arterial velocity displacement data 38. The method of arterial tissue Doppler imaging (TDI) is used to measure the low velocity, high amplitude signals created by the tissue. Arterial displacement data 38 are acquired using tissue-specific presets programmable in the ultrasound system (AWM preset; ATL5000, Philips/ATL Bothell Wash., USA) to determine frame rate, image size, and pre- and post-processing values. When a sufficient area of the carotid artery of the patient 27 is seen, usually 2-10 cm from the bifurcation, the area is zoomed in 2D and then a similar color Doppler zoom box is superimposed on the patient's artery 27 to cover the outer edges of the adventitia and surrounding tissue. Color gain is set to 100%, focus is set in or about the far (posterior) wall of the patient's artery 27 and the highest frame rate possible is achieved (usually 140-200 frames per second). Arterial displacement data images 38 are acquired as digital cine-loops consisting of 3-5 cardiac cycles and stored to 3.5″ optical disk for off-line analysis. The best quality image between the anterior, lateral and posterior views is selected for use for image acquisition.

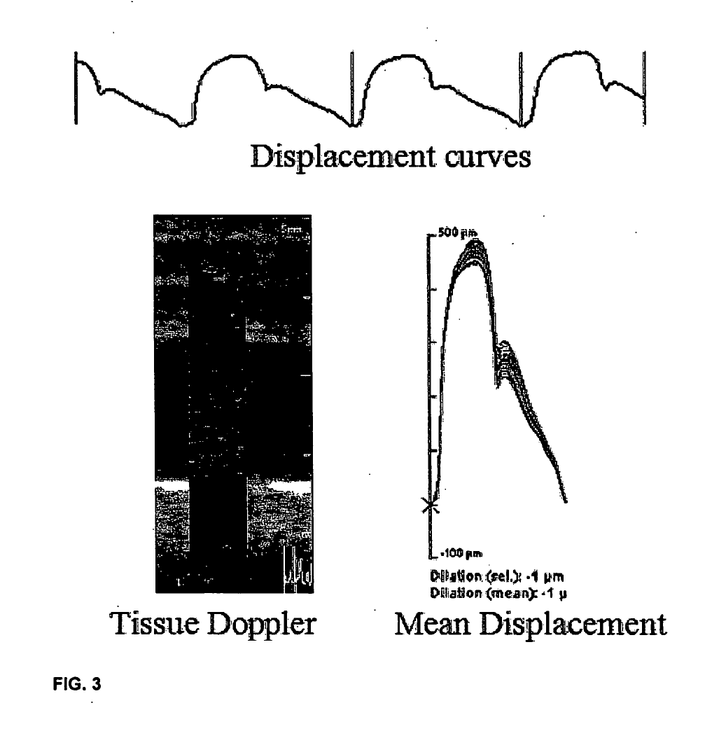

Arterial velocity displacement data 38 are adjusted off-line using software programs 40 which integrate velocity with respect to time. A suitable software program 40 (eg. Arterial Wall Motion v2.0 (AWM), Philips/ATL, Bothell Wash., USA) plots the arterial wall velocities of the entire colour Doppler sector over the cardiac cycles to reconstruct a central pressure waveform or adjusted arterial velocity displacement data 42 thereby generating quantitative measurements from the arterial Doppler imaging velocity data (obtained from TDI) 38 for arterial displacement (μm) over time as shown in FIG. 3. These adjusted arterial velocity displacement data 42 can then be exported in the format of a readable spreadsheet for further software analysis, eg. as csv or xls file formats.

In the preferred embodiment, the adjusted arterial velocity displacement data 42 are imported into a software program 44 custom written in MatLab (eg. Samtdi v1.0 SG Carlier).

In one embodiment, blood pressure data 46 are acquired from the patient 26 using a manometer 48 or any like pressure reading device known in the art. Preferably, the blood pressure data 46 acquired is mean (2×diastolic BP+systolic BP/3) and diastolic brachial cuff blood pressure.

Adjusted velocity displacement data 42 are calibrated 49 using software 44 with respect to cuff blood pressure data 46, so that the resulting arterial displacement waveform data 50 is calibrated for blood pressure 48. Significantly, the only previous work involving the use of colour tissue Doppler for this purpose did not consider or calibrate for cuff blood pressure, which clearly influences distensibility.

In another embodiment, the adjusted velocity displacement data 42 are corrected 51 using software 44 for the generation of values for local arterial elasticity 28 and other haemodynamic measures using the Doppler and pressure data. The correction is generated by dividing the observed displacement data by the log of the pulse pressure obtained from cuff BP.

FIG. 3 shows the output from the analysed arterial colour tissue Doppler with raw tissue Doppler (lower left), individual displacement curves for each cardiac cycle (top), and mean displacement curve (bottom right).

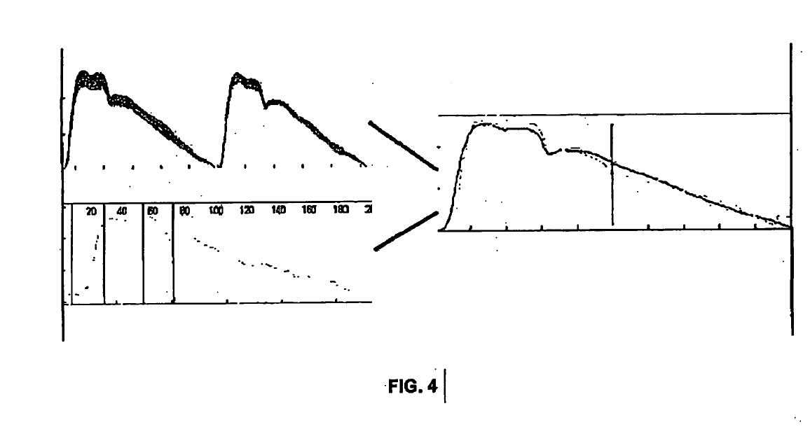

Noteworthy, the resulting arterial displacement data 28 (FIG. 4) is analogous to that obtained by tonometry, however, rather than reflecting systemic blood pressure, the velocity waveform data 28 advantageously reflects the local behaviour of the vessel wall. Furthermore, this new vascular imaging method 10 eliminates the need of using a radial-aortic transfer function, as is required with radial tonometry.

It will be appreciated that the arterial displacement data 28 provides new information about elastic vessels which is not provided by known tests, but rather reflect endothelial function and systemic (i.e. rather than local) compliance.

Advantageously, this novel ultrasound-based method 10 can be readily loaded as software onto existing echo-Doppler machines 32, 34 for acquisition of TDI image data 38, for which cardiologists and physicians with vascular interests are familiar and already use widely. The analysis software 40, 44 too may be easily loaded onto a PC for off-line analysis.

By way of the following examples, the present apparatus and method provides means of assessing arterial distensibility as a measure of cardiovascular disease progression.

### EXAMPLE 1

### Ability to Distinguish Groups with Different Degrees of Arterial Disease

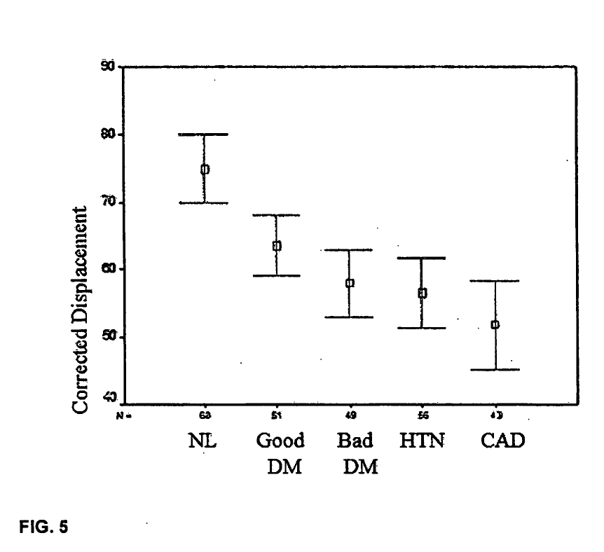

The ability to distinguish groups with different degrees of arterial disease was demonstrated in a study of >220 patients having various risk factors. Normal patients were compared with those with uncomplicated diabetes (good Diabetes Mellitus or “good DM”), DM and complications (“bad DM”), hypertension and known coronary disease (CAD). As shown in FIG. 5, as the severity of the vascular disease increased, so also did carotid distensibility. This remained so even after displacement was corrected for increased vessel size with increased severity of disease, as well as pulse pressure. Thus, it can be readily seen that carotid TDI, when calibrated using cuff BP provides an effective test for sub-clinical arterial disease as it detects increasing distensibility as a function of increasing vessel damage.

### EXAMPLE 2

### Comparison with Existing Techniques for Assessment of Arterial Distensibility

1. Total Arterial Compliance (TAC)

Various tests are known to measure arterial distensibility. The measurement of total arterial compliance (TAC) is widely considered the most suitable as this pulse pressure method 10 is able to incorporate stroke volume, which has an important influence on compliance. Specifically, the compliance method used by the inventors is derived from tonometry measurement at the radial pulse, use of a transfer function to obtain central pressure and Doppler measurement of stroke volume.

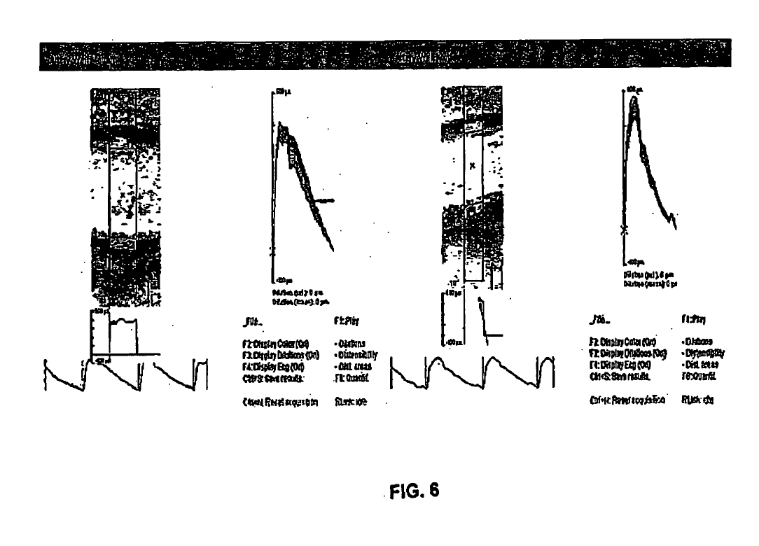

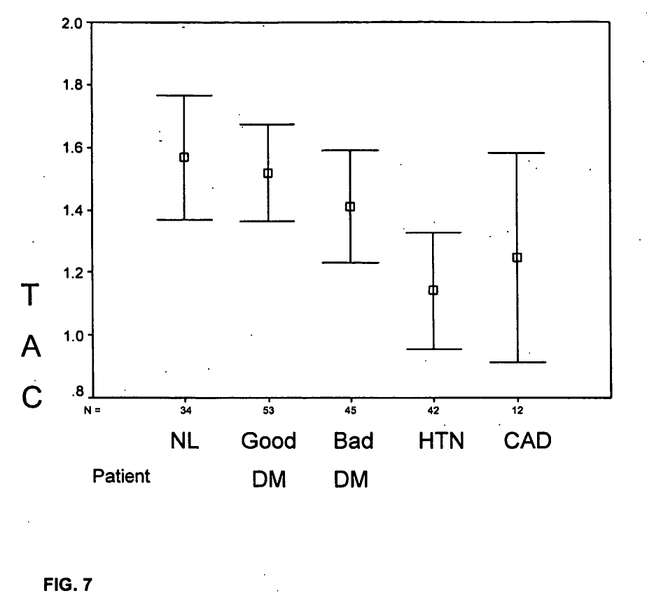

The inventors compared compliance measurements used by various groups with that of the compliance methods 10 of the present invention. Broad correlation was found between compliance and distensibility (r=0.52, p<0.001), as anticipated from two measures of arterial function. FIG. 6 illustrates a study of two patients: FIG. 6A (left hand side) has highly compliant arteries and high displacement (450 microns) at a low BP and, in contrast, FIG. 6B (right hand side) shows a patient with reduced compliance, and less displacement (349 microns) even at a high BP. Significantly, the carotid distensibility measures only the behaviour of the carotid, a large and mainly elastic artery that might be damaged, particularly in hypertension. However, while compliance was found abnormal in patients with severe disease (see FIG. 7), in contrast with arterial displacement (see FIG. 5), compliance shows less distinction between normal and abnormal.

2. Carotid Intima-Medial Thickness (IMT)

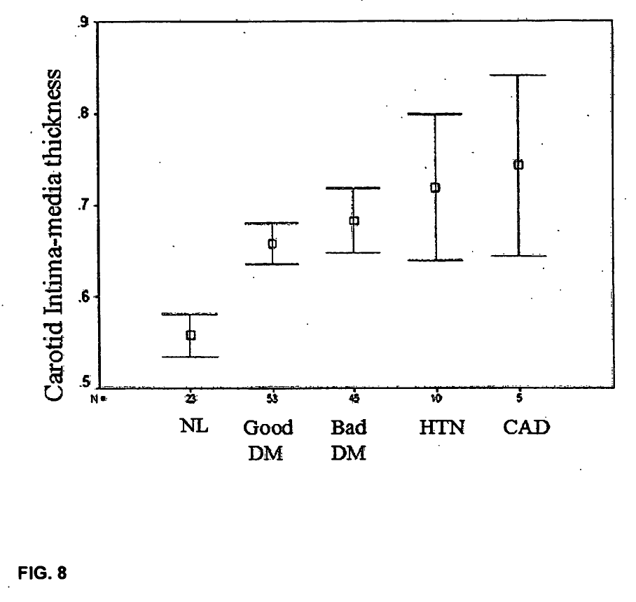

Measurement of intima-medial thickness (IMT) is a direct anatomic measurement of arterial thickening, including both intima (atheroma) and media (hypertensive disease). The correspondence between IMT and subgroups of patients with progressing disease is shown in FIG. 8. An increase in IMT corresponds to lower arterial displacement. The correlation between the measurements is low, eg. 0.11, suggesting that different aspects of arterial disease are being measured.

3. Brachial Reactivity

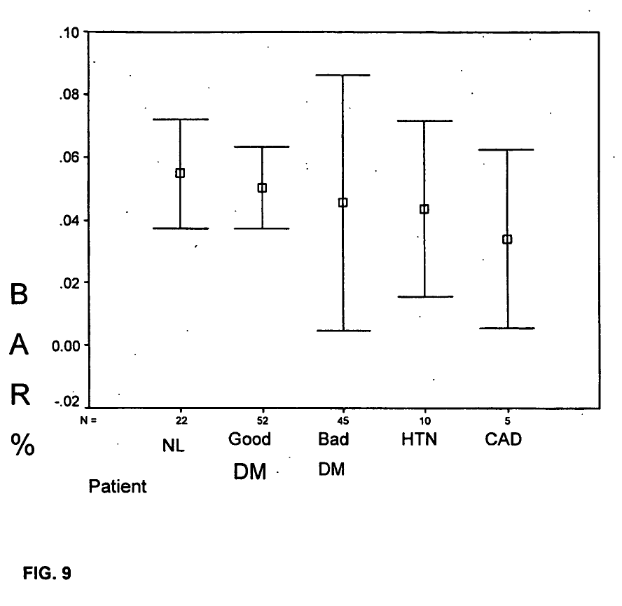

Measurement of brachial reactivity is a measure of the ability of the artery to dilate in response to hyperemia. This process is mediated by nitric oxide release from the endothelium and is influenced by a number of acute phenomena, including diet, stress etc. Accordingly, observations may result reflect these variations. Significantly, the process does not vary on the basis of increasing degrees of anticipated arterial damage (see FIG. 9).

Method Validation Studies

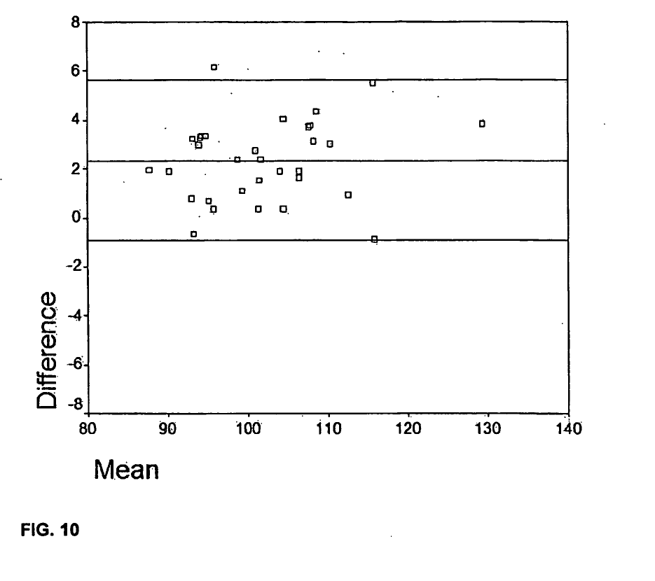

In order to demonstrate the efficacy of the invention, the inventors have conducted a validation study. FIG. 10 illustrates the difference between central systolic BP using tonometry and TDI in normal subjects. The average difference (y axis) over a range of systolic pressure (x axis) was 2 mmHg, thus demonstrating that central BP can be approximated using carotid TDI when calibrated using cuff BP.

Thus, it can be readily seen that the present invention provides a method and apparatus which demonstrates that (i) elasticity measurements using TDI are abnormal in pathologic states; (ii) elasticity measurements correspond to the physical properties of vessels; and (iii) elasticity measurement changes with therapies.

It will be appreciated that this novel method provides a validated, easily performed imaging technique which assesses arterial dysfunction which is suitable for use in facilitating the early diagnosis of vascular disease in those at risk. Further, this method is suitable for following a patient's response to therapy.

It will be appreciated by persons skilled in the art that the present invention is not limited to the embodiments described in detail herein, and that a variety of other embodiments may be contemplated which are nevertheless consistent with the broad spirit and scope of the invention.

1. A method for determining vascular characteristics for early detection of cardiovascular disease including the steps of:
(i) acquiring velocity displacement data from arterial colour tissue Doppler imaging;
(ii) processing the velocity displacement data to generate arterial displacement data;
(iii) adjusting the arterial displacement data using blood pressure data; and
(iv) analysing the adjusted arterial displacement data to characterise vascular function.

2. The method of claim 1 wherein the step of processing the velocity displacement data includes integrating velocity displacement data with respect to time.

3. The method of claim 1 wherein the step of processing the velocity displacement data includes using a readable spreadsheet for integrating velocity displacement data with respect to time.

4. The method of claim 1 wherein the step of adjusting the arterial displacement data includes dividing said arterial displacement data by a cuff blood pressure to obtain corrected displacement data.

5. The method of claim 1 wherein the step of analysing the adjusted arterial displacement data includes generating local elasticity data by correcting the arterial displacement data by dividing said arterial displacement data by a log of a cuff blood pressure.

6. An apparatus for determining vascular characteristics for early detection of cardiovascular disease comprising:
an ultrasonic signal source directing ultrasound signals at an artery;
an ultrasonic signal receiver receiving ultrasound signals reflected from or transmitted through the artery;
software for analysing signals received by the ultrasonic signal receiver to extract arterial displacement data;
software for adjusting said arterial displacement data using blood pressure data; and
software for analysing the adjusted arterial displacement data to characterise vascular function.

7. The apparatus of claim 6 wherein the software for analysing signals received by the ultrasonic signal receiver includes software for integrating velocity displacement data with respect to time.

8. The apparatus of claim 6 wherein the blood pressure data comprise diastolic and mean brachial cuff blood pressure data.

9. The apparatus of claim 6 wherein the blood pressure data are acquired using a manometer for measuring diastolic and mean brachial cuff blood pressure data.

10. The apparatus of claim 6 wherein the software for analysing the adjusted arterial displacement data includes software for generating vascular function data in the form of local elasticity data.

11. The apparatus of claim 10 wherein the software for generating vascular function data includes software for correcting adjusted arterial displacement data with a log of the blood pressure data.

12-13. (canceled)

14. An apparatus for determining vascular characteristics for early detection of cardiovascular disease, comprising:
an ultrasonic signal source for directing ultrasound signals at an artery;
an ultrasonic signal receiver for receiving ultrasound signals reflected from or transmitted through the artery; and
a processor operatively coupled to the ultrasonic signal receiver for:
analysing signals received by the ultrasonic signal receiver to extract arterial displacement data;
adjusting said arterial displacement data using blood pressure data; and
analysing adjusted arterial displacement data to characterise vascular function.

15. The apparatus of claim 14 wherein analysing signals received by the ultrasonic signal receiver comprises integrating velocity displacement data with respect to time.

16. The apparatus of claim 14 wherein the blood pressure data comprise diastolic and mean brachial cuff blood pressure data.

17. The apparatus of claim 14 further comprising a manometer operatively coupled to the processor for measuring diastolic and mean brachial cuff blood pressure data.

18. The apparatus of claim 14 wherein analysing the adjusted arterial displacement data comprises generating vascular function data in the form of local elasticity data.

19. The apparatus of claim 18 wherein generating vascular function data comprises correcting adjusted arterial displacement data with a log of cuff blood pressure data.
